SEQUENCE LISTING (22 sequences)

SEQ ID NO: 1 - protein, 118 aa, Artificial
  Synthetic Construct
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 2 - protein, 107 aa, Artificial
  Synthetic Construct
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 3 - protein, 5 aa, Artificial
  Synthetic Construct
SYYMH

SEQ ID NO: 4 - protein, 7 aa, Artificial
  Synthetic Construct
GYTFTSY

SEQ ID NO: 5 - protein, 10 aa, Artificial
  Synthetic Construct
GYTFTSYYMH

SEQ ID NO: 6 - protein, 17 aa, Artificial
  Synthetic Construct
EISPFGGRTNYNEKFKS

SEQ ID NO: 7 - protein, 7 aa, Artificial
  Synthetic Construct
ISPFGGR

SEQ ID NO: 8 - protein, 9 aa, Artificial
  Synthetic Construct
ERPLYASDL

SEQ ID NO: 9 - protein, 11 aa, Artificial
  Synthetic Construct
RASQGISSALA

SEQ ID NO: 10 - protein, 7 aa, Artificial
  Synthetic Construct
SASYRYT

SEQ ID NO: 11 - protein, 9 aa, Artificial
  Synthetic Construct
QQRYSLWRT

SEQ ID NO: 12 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 13 - protein, 117 aa, Artificial
  Synthetic Construct
EVQLVESGGGLVKPGGSLRLSCAASGFTFDDSVMHWVRQAPGKGLEWVSLVGWDGFFTYYADSVKGRFTISRDNAKNSLYLQMNSLRAEDTAVYYCARQGDYMGNNWGQGTLVTVSS

SEQ ID NO: 14 - protein, 110 aa, Artificial
  Synthetic Construct
NFMLTQPHSVSESPGKTVTISCTRSSGSIDSSYVQWYQQRPGSSPTTVIYEDDQRPSGVPDRFSGSIDSSSNSASLTISGLKTEDEADYYCQSYDFHHLVFGGGTKLTVL

SEQ ID NO: 15 - protein, 432 aa, Artificial
  Synthetic Construct
EVQLVESGGGLVKPGGSLRLSCAASGFTFDDSVMHWVRQAPGKGLEWVSLVGWDGFFTYYADSVKGRFTISRDNAKNSLYLQMNSLRAEDTAVYYCARQGDYMGNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 215 aa, Artificial
  Synthetic Construct
NFMLTQPHSVSESPGKTVTISCTRSSGSIDSSYVQWYQQRPGSSPTTVIYEDDQRPSGVPDRFSGSIDSSSNSASLTISGLKTEDEADYYCQSYDFHHLVFGGGTKLTVLQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - protein, 5 aa, Artificial
  Synthetic Construct
DSVMH

SEQ ID NO: 18 - protein, 17 aa, Artificial
  Synthetic Construct
LVGWDGFFTYYADSVKG

SEQ ID NO: 19 - protein, 8 aa, Artificial
  Synthetic Construct
QGDYMGNN

SEQ ID NO: 20 - protein, 13 aa, Artificial
  Synthetic Construct
TRSSGSIDSSYVQ

SEQ ID NO: 21 - protein, 7 aa, Artificial
  Synthetic Construct
EDDQRPS

SEQ ID NO: 22 - protein, 9 aa, Artificial
  Synthetic Construct
QSYDFHHLV

(19) United States
(12) Patent Application Publication
(10) Pub. No.: US 2023/0134160 A1
(43) Pub. Date: May 4, 2023

(54) Method Of Preparing A Therapeutic Protein Formulation And Antibody Formulation Produced By Such A Method

(71) Applicant: PFIZER INC., New York, NY (US)

(72) Inventors: Judy Kay GLYNN, Columbia, IL (US); Brian Xin CHEN, Chesterfield, MO (US); Daniel Patrick LACASSE, Leominster, MA (US)

(73) Assignee: PFIZER INC., New York, NY (US)

(21) Appl. No.: 15/761,777

(22) PCT Filed: Sep. 8, 2016
(86) PCT No.: PCT/IB2016/055355

§ 371 Date: Mar. 20, 2018

Related U.S. Application Data
(60) Provisional application No. 62/222,067, filed on Sep. 22, 2015.

(51) Int. Cl. A61K 39/395; C07K 1/34; A61K 47/22; A61K 47/26; A61K 47/18; C07K 16/28

(52) U.S. Cl. CPC A61K 39/39591; C07K 1/34; A61K 47/22; A61K 47/26; A61K 47/183; C07K 16/2866

(57) ABSTRACT

The invention relates to a method of preparing a protein formulation including a therapeutic protein, the method comprising the steps of: providing a solution comprising said protein; concentrating the protein in the solution by a first ultra-filtration step; diafiltering the solution with a diafiltration buffer including at least one first excipient, whereby a retentate is obtained comprising the protein and the first excipient; further concentrating the protein in the retentate by a second ultra-filtration step; and adding at least one final excipient, whereby the protein formulation with a desired protein concentration is obtained. According to the inven tion, the method further comprises, before the second ultra-filtration step, adding a second excipient to the retentate obtained from the diafiltration step. The invention is also directed to antibody formulations produced by the foregoing method.

This application is a § 371 filing of PCT/IB2016/055355 filed Sep. 8, 2016, which claims the benefit of priority to U.S. Provisional Application No. 62/222,067 filed Sep. 22, 2015; the entire contents of which are incorporated herein by reference.

### REFERENCE TO SEQUENCE LISTING

This application is being filed electronically via EFS-Web and includes an electronically submitted sequence listing in .txt format. The .txt file contains a sequence listing entitled “PC72213A_SEQ_LST_ST25.txt” created on Mar. 13, 2018, and having a size of 19 KB. The sequence listing contained in this .txt file is part of the specification and is herein incorporated by reference in its entirety.

### TECHNICAL FIELD

The present invention relates to a method of preparation of a protein formulation including excipients and at least one therapeutic protein.

The invention is of particular interest in the field of antibody formulations intended for a therapeutic use and is also directed to an antibody formulation produced by the method.

### BACKGROUND OF THE INVENTION

The invention is more particularly related to methods sequentially comprising:
providing a solution comprising said protein;
concentrating the protein in the solution by a first ultra-filtration step;
diafiltering the solution thus obtained with a diafiltration buffer including at least one first excipient, whereby a retentate is obtained comprising the protein and the first excipient;
further concentrating the protein in the retentate by a second ultra-filtration step in an ultra-filtration equipment;
adding at least one final excipient, whereby the protein formulation with a desired protein concentration and including said first and final excipients is obtained.

In general, the final protein formulations for therapeutic antibodies include at least an amino-acid, such as histidine, which is added during the diafiltration step, and a sugar acting as a stabilizer, such as trehalose. The trehalose is commonly added with the other excipients in the final addition step.

In conventional methods applied to therapeutic antibodies, the above steps are performed with a protein solution, once purified by a number of purification steps usually including a virus retaining filtration as the last purification step. The protein solution (or “product”) is concentrated by the first ultra-filtration step, from a concentration of approximately 5 to 20 g/l to a concentration of about 40 to 100 g/l (depending on the protein). Then the concentrated product is diafiltered in a diafiltration buffer, such as histidine. In some instances, the diafiltration buffer may be another standard buffer such as acetate, tris or phosphate. The diafiltration buffer is chosen based on the final protein formulation as well as on any offset that is required due to the Donnan effect. The Donnan effect occurs as the product is concentrated and results in the exclusion of certain charged buffer species, e.g. histidine. The diafiltration buffer is therefore usually adjusted to a higher buffer concentration and a lower pH than are specified for the protein formulation. Once the diafiltration is complete, the product goes through the second ultra-filtration step for concentration to approximately 50% above the desired protein concentration for the final protein formulation. Then the product is removed from the ultra-filtration system and the system is rinsed to recover additional product. With a final concentration of more than 50% above the desired concentration in the protein formulation, all of the rinse can be added back to the product to maximize recovery, without excessively diluting the product. Then the excipients are added (sugar, surfactant, chelator, etc.) as a concentrated solution, usually with a dilution ratio of approximately 4, meaning that 1 unitary volume of the concentrated excipient solution is added to 3 unitary volumes of the product. The dilution ratio of 4 is based on the maximum solubility of the sugar component of the excipient solution, which is usually the limiting factor. If necessary, the product is then further diluted with formulation buffer for adjustment to the final desired concentration.

Such conventional methods may therefore not be applicable when it is desired to obtain a highly concentrated protein in the final formulation, and even more when the protein is of particularly high viscosity.

For example, in the case of a therapeutic antibody formulation with a desired final concentration of 150 g/l, the viscosity of the molecule precludes concentrating to the targeted value of 50% above the desired final concentration.

It is an aim of the invention to provide a method of preparation of a protein formulation that may be applied to highly viscous and highly concentrated proteins.

It is a further aim of the invention that the method may be implemented at a manufacturing scale, without negatively affecting the overall yield of the manufacturing process and without incurring extra costs due to an excessive waste of certain excipients. In particular, it is an aim to keep the use of the sugar components, which are particularly costly, at a similar level as the conventional methods.

It is still a further aim to preserve the stability of the protein over all the steps of the method and to protect the protein from aggregation.

### SUMMARY

According to a first aspect of the present invention, there is provided a method of the above type further comprising, before the second ultra-filtration step, adding a second excipient to the retentate obtained from the diafiltration step.

By moving the addition of a second excipient, in particular the trehalose (or more generally the sugar), to post-diafiltration, the remaining excipients can be added at a much higher concentration in a subsequent step, thus generating a lower dilution of the product. This in turn means that the maximal required concentration can be brought to only about 10% (in some instances between 5 to 15%) above the final desired concentration, as compared to the value of about 50% for the conventional methods. This 10% value is obtainable with standard ultra-filtration equipments, even with higher molecule viscosity. This also allows recovering product from a rinse and thus allows obtaining a 90% yield of the ultra-filtration/dia-filtration process.

Also, adding the second excipient (the sugar) before the final concentration protects the protein from aggregation.

According to preferred embodiments of the invention:
the method further includes, after step (e) and before step (f), rinsing the ultra-filtration equipment with a rinse buffer, whereby the recovery of the protein is enhanced;
the rinse buffer comprises the first and the second excipients at concentrations substantially equal to, respectively, the concentrations of the first and of the second excipients in the protein formulation;
the first excipient is an amino-acid, preferably histidine;
the first excipient in the protein formulation has a concentration of between 16 and 24 mM, preferably of between 17 and 23 mM, most preferably of about 20 mM;
the second excipient is a sugar, preferably a disaccharide;
the final excipients include a surfactant, preferably polysorbate 80;
the final excipients include a chelating agent, preferably EDTA;
the protein formulation has a protein concentration of between 110 and 165 g/l;
the protein is an antibody.

In a first preferred embodiment:
the antibody is an anti-PCSK9 (Proprotein Convertase Subtilisin Kexin type 9) antibody;
the anti-PCSK9 antibody is selected from the group consisting of bococizumab, evolocumab (REPATHATM), alirocumab (PRALUENT™), REGN728, 31H4, 11F1, 12H11, 8A1, 8A3, 3C4, 300N, 1D05, LGT209, RG7652, and LY3015014;
the anti-PCSK9 antibody comprises a heavy chain variable region (VH) comprising complementarity determining region one CDR1, CDR2, and CDR3 of the amino acid sequence shown in SEQ ID NO: 1; and a light chain variable region (VL) comprising CDR1, CDR2, and CDR3 of the amino acid sequence shown in SEQ ID NO: 2; or alternatively the anti-PCSK9 antibody comprises a VH CDR1 having the amino acid sequence shown in SEQ ID NO: 3, 4, or 5, a VH CDR2 having the amino acid sequence shown in SEQ ID NO: 6 or 7, a VH CDR3 having the amino acid sequence shown in SEQ ID NO: 8, a VL CDR1 having the amino acid sequence shown in SEQ ID NO: 9, a VL CDR2 having the amino acid sequence shown in SEQ ID NO:10, and a VL CDR3 having the amino acid sequence shown in SEQ ID NO: 11;
the protein formulation has a protein concentration of between 135 and 165 g/l, preferably of between 142 and 158 g/l, most preferably of about 150 g/l;
the second excipient in the protein formulation is trehalose at a concentration of between 67.2 and 100.8 g/l, preferably of between 71.4 and 96.6 g/l, most preferably of about 84 g/l;
the final excipients include polysorbate 80 which, in the protein formulation, has a concentration of between 0.16 and 0.24 g/l, preferably of between 0.17 and 0.23 g/l, most preferably of about 0.2 g/l;
the final excipients include EDTA which, in the protein formulation, has a concentration of between 0.04 and 0.06 g/l, preferably of between 0.0425 and 0.0575 g/l, most preferably of about 0.05 g/l;
the protein formulation has a pH of between 5.2 and 5.8, preferably of about 5.5;
the solution provided in step (a) has a protein concentration of between 5 and 20 g/l;
the protein is concentrated to between 80 and 120 g/l, preferably to between 90 and 110 g/l, and most preferably to about 100 g/l, by the first ultra-filtration step;
the protein is concentrated to between 143 and 173 g/l, preferably to between 150 and 166 g/l, and most preferably to about 158 g/l, by the second ultra-filtration step;
the first excipient in the diafiltration buffer has a concentration higher than the concentration of the first excipient in the protein formulation, said concentration of the first excipient in the diafiltration buffer being preferably of between 29.75 and 40.25 mM, most preferably of about 35 mM;
the diafiltration buffer has a pH of between 5.1 and 5.5, preferably about 5.3;
adding the second excipient to the retentate obtained from the diafiltration step is achieved by adding a first additive solution to the retentate, said first additive solution comprising the second excipient at a concentration of between 340 and 460 g/l, preferably of between 380 and 420 g/l, most preferably of about 400 g/l;
the first additive solution comprises the first excipient at a concentration lower than the concentration of the first excipient in the diafiltration buffer and higher than the concentration of the first excipient in the protein formulation, said concentration of the first excipient in the first additive solution being preferably of between 25.5 and 34.5 mM, most preferably of about 30 mM;
the first additive solution further comprises a final excipient;
the first additive solution comprises about 30 mM histidine and about 400 g/l trehalose;
adding the first additive solution to the retentate is performed at a dilution ratio of about 4.15, whereby one volume of the first additive solution is added to approximately 3.15 fold the same volume of the retentate;
adding the final excipients includes the step of adding a second additive solution to the solution obtained from the second ultra-filtration step, said second additive solution comprising the second excipient at a concentration lower than the concentration of the second excipient in the first additive solution and higher than the concentration of the second excipient in the protein formulation;
the second additive solution comprises the first excipient at a concentration substantially equal to the concentration of the first excipient in the protein formulation;
the second additive solution comprises about 20 mM histidine, about 84 g/l trehalose, about 1 g/l EDTA and about 4 g/l polysorbate 80;
adding the second additive solution is performed at a dilution ratio of about 20, whereby one volume of the second additive solution is added to approximately 19 fold the same volume of to the solution obtained from the second ultra-filtration step.

In a second preferred embodiment:
the antibody is an anti-IL7R antibody;
preferably, the anti-IL-7R antibody comprises a heavy chain variable region (VH) comprising complementarity determining region one CDR1, CDR2, and CDR3 of the amino acid sequence shown in SEQ ID NO: 13 (examples of the sequences of such CDRs are SEQ ID NOs. 17, 18 and 19 respectively); and a light chain variable region (VL) comprising CDR1, CDR2, and CDR3 of the amino acid sequence shown in SEQ ID NO: 14 (examples of the sequences of such CDRs are SEQ ID Nos. 20, 21 and 22 respectively);
more preferably, the VH region of the anti-IL-7R antibody comprises the amino acid sequence shown in SEQ ID NO. 13, and the VL region of the anti-IL-7R antibody comprises the amino acid sequence shown in SEQ ID NO. 14;
even more preferably, the heavy chain of the anti IL-7R antibody comprises the amino acid sequence shown in SEQ ID NO. 15 and a light chain of the anti IL-17 antibody has the amino acid sequence shown in SEQ ID NO. 16;
the protein formulation has a protein concentration of between 110 and 130 g/l, preferably of about 120 g/l;
the second excipient in the protein formulation is sucrose at a concentration of between 42 and 58 g/l, preferably of about 50 g/l;
the final excipients include polysorbate 80 which, in the protein formulation, has a concentration of between 0.017 and 0.023 g/l, preferably of about 0.02 g/l;
the final excipients include EDTA which, in the protein formulation, has a concentration of between 0.42 and 0.58 g/l, preferably of about 0.5 g/l;
the final excipients include arginine which, in the protein formulation, has a concentration of between 85 and 115 mM, preferably of about 100 mM;
the protein formulation has a pH of between 6.5 and 7.5, preferably of about 7.0;
the solution provided in step (a) has a protein concentration of between 2.6 and 3.4 g/l, preferably of about 3 g/l;
the protein is concentrated to between 36 and 54 g/l, preferably to between 40 and 50 g/l, and most preferably to about 45 g/l, by the first ultra-filtration step;
the protein is concentrated to between 170 and 210 g/l, preferably to about 190 g/l, by the second ultra-filtration step;
the first excipient in the diafiltration buffer has a concentration higher than the concentration of the first excipient in the protein formulation, said concentration of the first excipient in the diafiltration buffer being preferably of between 19 and 25 mM, most preferably of about 22 mM;
the diafiltration buffer includes arginine at a concentration of between 95 and 125 mM, preferably of about 110 mM;
the diafiltration buffer has a pH of between 6.5 and 7.5, preferably about 7.0;
adding the second excipient to the retentate obtained from the diafiltration step is achieved by adding a first additive solution to the retentate, said first additive solution comprising the second excipient at a concentration of between 230 and 320 g/l, preferably of about 275 g/l;
the first additive solution comprises the first excipient at a concentration substantially equal to the concentration of the first excipient in the diafiltration buffer and higher than the concentration of the first excipient in the protein formulation, said concentration of the first excipient in the first additive solution being preferably of between 19 and 25 mM, most preferably of about 22 mM;
the first additive solution further comprises a final excipient;
the first additive solution comprises about 22 mM histidine, 110 mM arginine and about 275 g/l sucrose, at a pH of about 7.0;
adding the first additive solution to the retentate is performed at a dilution ratio of about 5, whereby one volume of the first additive solution is added to approximately 4 fold the same volume of the retentate;
adding the final excipients includes the step of adding a second additive solution to the solution obtained from the second ultra-filtration step, said second additive solution comprising EDTA and polysorbate 80;
adding the second additive solution is performed at a dilution ratio of about 20, whereby one volume of the second additive solution is added to approximately 19 fold the same volume of to the solution obtained from the second ultra-filtration step.

According to a second aspect of the invention, there is provided an antibody formulation produced by the foregoing method.

In a preferred embodiment, the protein formulation comprises:
from 135 mg/ml to 165 mg/ml, preferably about 150 mg/ml, of the anti-PCSK9 antibody, and
from 16 mM to 24 mM, preferably about 20 mM, of histidine buffer.

In another preferred embodiment, the protein formulation comprises:
from 135 mg/ml to 165 mg/ml, preferably about 150 mg/ml, of the anti-PCSK9 antibody, and
from 67.2 mg/ml to 100.8 mg/ml, preferably about 84 mg/ml, of trehalose.

In another preferred embodiment, the protein formulation comprises:
from 135 mg/ml to 165 mg/ml, preferably about 150 mg/ml, of the anti-PCSK9 antibody, and
from 0.16 mg/ml to 0.24 mg/ml, preferably about 0.2 mg/ml, of polysorbate.

In another preferred embodiment, the protein formulation comprises:
from 135 mg/ml to 165 mg/ml, preferably about 150 mg/ml, of the anti-PCSK9 antibody,
from 16 mM to 24 mM, preferably about 20 mM, of histidine buffer, and
from 67.2 mg/ml to 100.8 mg/ml, preferably about 84 mg/ml, of trehalose.

In another preferred embodiment, the protein formulation comprises:
from 135 mg/ml to 165 mg/ml, preferably about 150 mg/ml, of the anti-PCSK9 antibody,
from 16 mM to 24 mM, preferably about 20 mM, of histidine buffer, and
from 0.16 mg/ml to 0.24 mg/ml, preferably about 0.2 mg/ml, of polysorbate.

In another preferred embodiment, the protein formulation comprises:
from 135 mg/ml to 165 mg/ml, preferably about 150 mg/ml, of the anti-PCSK9 antibody,
from 67.2 mg/ml to 100.8 mg/ml, preferably about 84 mg/ml, of trehalose, and
from 0.16 mg/ml to 0.24 mg/ml, preferably about 0.2 mg/ml, of polysorbate.

In a still preferred embodiment, the protein formulation comprises:
from 135 mg/ml to 165 mg/ml, preferably about 150 mg/ml, of the anti-PCSK9 antibody,
from 16 mM to 24 mM, preferably about 20 mM, of histidine buffer,
from 67.2 mg/ml to 100.8 mg/ml, preferably about 84 mg/ml, of trehalose, and
from 0.16 mg/ml to 0.24 mg/ml, preferably about 0.2 mg/ml, of polysorbate.

In some embodiments, the antibody formulation has a pH of between 5.2 and 5.8, preferably about 5.5.

In another preferred embodiment, the protein formulation comprises:
from 110 g/l to 130 g/l, preferably about 120 g/l, of the anti-IL-7R antibody;
from 17 mM to 23 mM, preferably about 20 mM, of histidine;
from 42 g/l to 58 g/l, preferably about 50 g/l, of sucrose; and
from 0.017 g/l to 0.023 g/l, preferably about 0.02 g/l, of polysorbate and has a pH of between 6.5 and 7.5, preferably about 7.0.

SEQ ID NO: 1 to 12 referred to in the foregoing are described in the table below:

| | |
|---|---|
| 1 | qvqlvqsgae vkkpgasvkv sckasgytft syymhwvrqa pgqglewmge |
| (VH) | ispfggrtny |
| | nekfksrvtm trdtststvy melsslrsed tavyycarer plvasdlwgq gttvtvss |
| 2 | diqmtqspss lsasvgdrvt itcrasqgis salawyqqkp gkapklliys asyrytgvps |
| (VL) | rfsgsgsgtd ftftisslqp ediatyycqq ryslwrtfgq gtkleik |
| 3 | SYYMH |
| (VH-CDR1) | |
| 4 | GYTFTSY |
| (VH-CDR1) | |
| 5 | GYTFTSYYMH |
| (VH-CDR1) | |
| 6 | EISPFGGRTNYNEKFKS |
| (VH-CDR2) | |
| 7 | ISPFGGR |
| (VH-CDR2) | |
| 8 | ERPLYASDL |
| (VH-CDR3) | |
| 9 | RASQGISSALA |
| (VL-CDR1) | |
| 10 | SASYRYT |
| (VL-CDR2) | |
| 11 | QQRYSLWRT |
| (VL-CDR3) | |
| 12 | MGTVSSRRSW WPLPLLLLLL LLLGPAGARA QEDEDGDYEE |
| (PCSK9 human) | LVLALRSEED GLAEAPEHGT TATFHRCAKD PWRLPGTYVV |
| | VLKEETHLSQ SERTARRLQA QAARRGYLTK ILHVFHGLLP |
| | GFLVKMSGDL LELALKLPHV DYIEEDSSVF AQSIPWNLER |
| | ITPPRYRADE YQPPDGGSLVEVYLLDTSIQ SDHREIEGRV |
| | MVTDFENVPE EDGTRFHRQA SKCDSHGTHLAGVVSGRDAG |
| | VAKGASMRSL RVLNCQGKGT VSGTLIGLEF IRKSQLVQPV |
| | GPLVVLLPLA GGYSRVLNAA CQRLARAGW LVTAAGNFRD |
| | DACLYSPASA PEVITVGATN AQDQPVTLGT LGTNFGRCVD |
| | LFAPGEDIIG ASSDCSTCFV SQSGTSQAAA HVAGIAAMML |
| | SAEPELTLAE LRQRLIHFSAKDVINEAWFP EDQRVLTPNL |
| | VAALPPSTHG AGWQLFCRTV WSAHSGPTRM ATAVARCAPD |
| | EELLSCSSFS RSGKRRGERM EAQGGKLVCR AHNAFGGEGV |
| | YAIARCCLLP QANCSVHTAPPAEASMGTRV HCHQQGHVLT |
| | GCSSHWEVED LGTHKPPVLR PRGQPNQCVG HREASIHASC |
| | CHAPGLECKV KEHGIPAPQE QVTVACEEGW TLTGCSALPG |
| | TSHVLGAYAV DNTCVVRSRDVSTTGSTSEG AVTAVAICCR |
| | SRHLAQASQE LQ |

SEQ ID Nos. 13-16 in the forgoing are described in the table below

| | |
|---|---|
| 13 | EVQLVESGGGLVKPGGSLRLSCAASGFTFDDSVMHWVRQAPGKGLEWVSLV |
| (VH) | GWDGFFTYYADSVKGRFTISRDNAKNSLYLQMNSLRAEDTAVYYCARQGDYM |
| | GNNWGQGTLVTVSS |
| 14 | NFMLTQPHSVSESPGKTVTISCTRSSGSIDSSYVQWYQQRPGSSPTTVI |
| (VL) | YEDDQRPSGVPDRFSGSIDSSSNSASLTISGLKTEDEADYYCQSYDFHH |
| | LVFGGGTKLTVL |
| 15 | EVQLVESGGGLVKPGGSLRLSCAASGFTFDDSVMHWVRQAPGKGLEW |
| | VSLVGWDGFFTYYADSVKGRFTISRDNAKNSLYLQMNSLRAEDTAVYYC |
| | ARQGDYMGNNWGQGTLVTVSSASTKGPSVFPLAPSSKSTSGGTAALG |
| | CLVKDYFPEPVTVSWNSGALTSGVHTFPAVLQSSGLYSLSSWTVPSSS |
| | LGTQTYICNVNHKPSNTKVDKKVAPELLGGPSVFLFPPKPKDTLMISRTP |
| | EVTCVVVDVSHEDPEVKFNWYVDGVEVHNAKTKPREEQYNSTYRVSV |
| | LTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| 16 | NFMLTQPHSVSESPGKTVTISCTRSSGSIDSSYVQWYQQRPGSSPTTVI |
| | YEDDQRPSGVPDRFSGSIDSSSNSASLTISGLKTEDEADYYCQSYDFHH |
| | LVFGGGTKLTVLQPKAAPSVTLFPPSSEELQANKATLVCLISDFYPGAVT |
| | VAWKADSSPVKAGVETTTPSKQSNNKYAASSYLSLTPEQWKSHRSYSC |
| | QVTHEGSTVEKTVAPTECS |
| 17 (VH-CDR1) | DSVMH |
| 18 (VH-CDR2) | LVGWDGFFTYYADSVKG |
| 19 (VH-CDR3) | QGDYMGNN |
| 20 (VL-CDR1) | TRSSGSIDSSYVQ |
| 21 (VL-CDR2) | EDDQRPS |
| 22 (VL-CDR3) | QSYDFHHLV |

### DETAILED DESCRIPTION

The following definitions will be used in the present description and claims:
the term “protein formulation” designates the final product including the protein of interest and excipients. When referring to proteins intended for a therapeutic use, the term “Drug Substance” may be used instead of “protein formulation” and the protein of interest may be designated by the term “active ingredient” or “product”. The “excipients” are defined by all the constituents of the “protein formulation”, which are not the “protein” or “active ingredient”. The excipients typically include protein stabilizers, surfactants, amino-acids e.g. contributing to protein stabilization, etc . . . ;
in connection with the dia-filtration step, the term “retentate” refers to the solution retained on the retentate side of the membrane and containing the molecules that are too large to pass through the membrane, such as the protein of interest. The retentate is the solution that is transferred to the subsequent part of the ultra-filtration/dia-filtration system. The other solution circulated on the other side (the permeate side) of the membrane in the dia-filtration part of the system is referred to as the “dia-filtration buffer” (or “basal buffer”);
the term “concentrated pool” designates the solution directly obtained from the final ultra-filtration step;
the term “final excipients” designates the excipients that are added to the “concentrated pool” after the final ultra-filtration step i.e. after the final concentration step;
the term “nx spike”, with n a numeral value, designates a solution of excipients that is added to a certain volume of the protein-containing solution, with a dilution ratio equal to n, which means that one volume of the solution of excipients is added to n−1 fold the same volume of the protein-containing solution. For example a 4× spike is a solution that is added according to the ratio: 1 volume of the spike for 3 volumes of the protein-containing solution;
unless stated otherwise, the terms “approximately”, “about” or “substantially” associated with a numeral value mean within a range of ±5% of said value;
“viscosity,” as used herein, may be “absolute viscosity” or “kinematic viscosity.” “Absolute viscosity,” sometimes called dynamic or simple viscosity, is a quantity that describes a fluid's resistance to flow. “Kinematic viscosity” is the quotient of absolute viscosity and fluid density. Kinematic viscosity is frequently reported when characterizing the resistive flow of a fluid using a capillary viscometer. When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. If both fluids have equal density, the second fluid is twice as viscous as the first on an absolute viscosity scale. The dimensions of kinematic viscosity are L²/T where L represents length and T represents time. The SI units of kinematic viscosity are m²/s. Commonly, kinematic viscosity is expressed in centistokes, cSt, which is equivalent to mm²/s. The dimensions of absolute viscosity are M/L/T, where M represents mass and L and T represent length and time, respectively. The SI units of absolute viscosity are Pas, which is equivalent to kg/m/s. The absolute viscosity is commonly expressed in units of centiPoise, cP, which is equivalent to milliPascal-second, mPa·s. In the context of the invention, an antibody is deemed to be of high viscosity if its viscosity is at least 20 cP.

For conciseness, the acronyms “UF”, “DF” and “UF/DF” (or “UFDF”) may be used across the description and should be understood as follows: “UF” means “ultra-filtration, “DF” means “dia-filtration” and “UF/DF” (or “UFDF”) means “ultra-filtration/dia-filtration”. The method of the invention, which is defined as a method of preparation of a protein formulation, may be referred to as a UFDF method.

The invention will now be further illustrated by the following Examples, each in connection with a specific therapeutic monoclonal antibody and a specific formulation of this monoclonal antibody. The Examples are provided for illustrative purpose only and should not be construed as limiting the scope of the invention.

### A—EXAMPLE 1

In illustrative Example 1, the protein of interest is bococizumab, a PCSK9-targeting monoclonal antibody that specifically binds to PCSK9 (Proprotein Convertase Subtilisin Kexin type 9), e.g. SEQ ID NO: 12 or Uniprot Accession Number Q8NBP7. The method has been designed to achieve a targeted product concentration of 150 g/l in the Drug Substance, with the Drug Substance including the following excipients at a pH of 5.5:
histidine at a 20 mM concentration,
trehalose at a 84 g/l concentration, and
PS80 (PolySorbate 80) at a 0.2 g/l concentration.

It is deemed acceptable that the above requirements are achieved with a tolerance of ±8 g/l in the protein concentration, of ±15% in the excipients concentration and of ±0.2 in the pH value.

In terms of yield, the method is required to achieve a product recovery of more than 90%.

Experiments have been conducted for defining preferred operating modes and establishing that the method of the invention is suitable for achieving the above requirements (while conventional methods are not). Some of these experiments are presented in the following part of the description.

A.1 Materials

The starting material used for experiments was a fully purified bococizumab solution that had been processed through a MabSelect® column to remove excipient components prior to use. After MabSelect® purification, the eluate was adjusted to pH 5.0 by acetic acid, resulting in a product concentration of 17.09 g/l.

Ultra-Filtration/Dia-Filtration Device

All experiments were performed using a GE Crossflow® system (300 ml reservoir) fitted with Pellicon® 3 (30 KDa, C-screen, 88 cm²) regenerated cellulose membranes or Sartocon® (30 KDa E-channel 200 cm²) regenerated cellulose membranes. TransMembrane Pressure (TMP) was maintained at approximately 14-22 psi with P_{Feed} less than 55 psi. Unless otherwise specified, all rinses were generated by recirculating rinse buffer for at least 15 minutes, then concentrating to the minimal working volume of the system.

A.2 Experimental Design and Results

Determination of Trehalose Solubility

An initial experiment was completed to evaluate the limit of the trehalose solubility in 30 mM histidine pH 5.35 solution (the histidine concentration and pH are adjusted from the final specifications to account for the exclusion of the histidine ion as the protein concentration increases). To obtain the 150 g/l final Drug Substance target, the minimal concentration of the concentrated pool would need to be 180 g/l with a 6× trehalose/EDTA/PS80 spike, or 187.5 g/l with a 5× trehalose/EDTA/PS80 spike. At the trehalose concentration required to provide a 6× spike (˜500 g/l), the trehalose did not dissolve (particulates were still present) at room temperature (22° C.) after extended stirring and had to be heated to 30° C. to dissolve. The solution was filtered through a 0.22 μm Pall Acrodisc® syringe filter under 15 psi pressure without re-precipitating at room temperature.

However, this manufacturing method may be difficult to scale up, therefore the maximum practical concentration for the trehalose spike may be capped at 5× (˜420 g/l trehalose).

Accordingly, in a preferred process, the trehalose concentration of the spike solution may be about 400 g/l.

Process Development

A first experiment was designed to test the histidine concentration needed in the dia-filtration solution, to check the histidine concentration in the dia-filtered solution at different protein concentrations (76.6 g/l and 114 g/l), and to generate material for density measurement. The starting material was concentrated to 76.6 g/l using a 200 cm² Sartocon® E-channel membrane at a load capacity of 345 g/m², and then dia-filtered with 35 mM histidine, pH 5.26 buffer. The flux of the dia-filtration was 17 LMH (liters/m²/hour) at 300 LMH feed flowrate and 22 psi TMP. The material was then further concentrated to 213 g/l (data not shown) and samples of both the diafiltered pool and final concentrated material were analyzed for histidine and trehalose concentration (see Table 1).

A second experiment was performed to determine if diafiltered material containing trehalose resulted in a lower final concentration versus material without trehalose in the diafiltration buffer. The starting material was concentrated to 114 g/L and diafiltered with 35 mM histidine, pH 5.26 buffer. The diafiltration flux was 10 LMH under the operational conditions described in Table 2, Experiment 2A. The diafiltered solution was concentrated to 184.9 g/L at <55 psi of feed pressure and 22 psi of TMP. The concentrated material was drained from the reservoir and combined with the 35 mM histidine, pH 5.26 rinse solution to achieve a concentration of 153.7 g/L. The pool was spiked with 4× trehalose excipient buffer (30 mM histidine, 400 g/L trehalose, pH 5.4) to achieve a final protein concentration of 114 g/L. The spiked solution was then concentrated to 202.4 g/L under the operational conditions described in Table 2, Experiment 2B. The concentration step was stopped at 15 LMH feed flow rate due to pump limitations.

Table 1 shows that both the histidine and trehalose concentrations in all concentrated samples were within 10% of the final target specification, 20 mM histidine and 84 g/L trehalose.

This information provides an acceptable operating range of the diafiltration concentration from 75-114 g/L, within which the final excipient concentrations meet concentration specifications.

TABLE 1
Initial Evaluation Excipient Concentration Results

| | Concentration | Histidine | Trehalose |
|---|---|---|---|
| Sample Name | (g/L) | (mM) | (g/L) |
| Diafiltration Exp 1 | 76.6 | 29.81 | Not Tested |
| Concentration Exp 1 | 213 | 19.63 | |
| Load Exp 2B | 114 | 26.19 | 83.82 |
| Concentration Exp2B | 202.4 | 18.67 | 76.37 |
| Diafiltration Buffer | N/A | 34.34 | Not Tested |

TABLE 2
Initial Evaluation Process Data

| UFDF System and Load | 706263-18-Exp2A | 706263-18-Exp2B |
|---|---|---|
| Charge | | |
| Membrane Type | Sartocon E-channel | |
| Membrane Area, m2 | 0.02 | 0.02 |
| UF Protein Challenge (g/m²) | 572 | 490 |
| Concentration 1 | | |
| Load Volume (ml) | 338.2 | NA |
| Load Concentration (g/L) | 33.8 | |
| Concentration 1 (g/L) | 114 | |
| Concentration 1 Process | Not recorded | |
| Time (hr) | | |
| Diafiltration | | |
| Diafiltration Buffer | 35 mM Histidine | NA |
| | pH 5.26 | |
| Diavolumes (TOV) | 8 | |
| Feed Flow Rate (LMH) | 300 | |
| Average Permeate Flux | 10 | |
| (LMH) | | |
| Average TMP (psi) | 22 | |
| Diafiltration Process | 4 | |
| Time (hr) | | |
| Concentration 2 | | |
| Spike Solution | NA | 30 mM Histidine, |
| | | 400 g/L trehalose |
| | | pH 5.4 |
| Post Spike Concentration | NA | 114 |
| (g/L) | | |
| Average TMP (psi) | 22 | 22 |
| Final Flow Rate (LMH) | Not recorded | 15 |
| Product Volume (ml) | 48.1 | Not recorded |
| Product Concentration (g/L) | 184.9 | 202.4 |
| Concentration 2 Process | Not recorded | ~1 |
| Time (hr)* | | |
| Retentate pH | 5.50 | 5.51 |
| Rinse | | |
| Rinse Buffer | 35 mM Histidine | 20 mM Histidine, |
| | pH 5.26 | 84 g/L trehalose |
| | | pH 5.5 |
| Rinse Volume (ml) | 26 | Not recorded |
| Rinse Concentration (g/L) | 58.7 | 54.5 |
| Rinse pH | Not recorded | 5.52 |

*The actual time was not recorded or could not be retrieved, it is based on a calculation of the flux and volume processed.

Additional experimentation was performed to evaluate changes in histidine and trehalose concentration as a function of protein concentration at end of the concentration 2 step. The starting material was concentrated to 105.9 g/l and dia-filtered with 35 mM histidine, pH 5.29 buffer using a 200 cm² Sartocon® E-channel to membrane. The flux of the dia-filtration was 12 LMH at a feed flow rate of 300 LMH and 22 psi TMP. The dia-filtered material was then spiked with 4× trehalose excipient solution. The spike solution was added directly into the reservoir and mixed for 15 minutes, then the material was concentrated to 172, 188 and 209 g/l final concentration (see Table 3). As shown in Table 4, the histidine concentration dropped as the protein concentration increased, but all values were within 10% of the target concentration of 20 mM histidine, 84 g/L trehalose.

TABLE 3
Additional Development Process Data

| UFDF System and Load Charge | Notebook: 706263-20 |
|---|---|
| Membrane Type | Sartocon Slice E-channel |
| Membrane Area, m2 | 0.02 |
| UF Protein Challenge (g/m²) | 477 |
| Concentration 1 | |
| Load Volume (ml) | 558.1 |
| Load Concentration (g/L) | 17.09 |
| Final Concentration (g/L) | 105.9 |
| Diafiltration | |
| Diafiltration Buffer | 35 mM Histidine pH 5.29 |
| Diavolumes (TOV) | 8 |
| Feed Flow Rate (LMH) | 300 |
| Average TMP (psi) | 22 |
| Average Permeate Flux (LMH) | 12 |
| Diafiltration Time (hr) | 3 |
| Concentration 2 | |
| Spike Solution | 30 mM Histidine, 400 g/L |
| | trehalose pH 5.22 |
| Post Spike Concentration (g/L) | 78.6 |
| Solution pH (At Spike) | 5.38 |
| Average TMP (psi) | 22 |
| Process Time (hr)* | ~1 |
| Product volume (ml) | 32.1 |
| Product Concentration (g/L) | 209 |
| Retentate pH | 5.53 |
| Yield Recovery (%) | 76.6 |
| Rinse | |
| Rinse Buffer | 20 mM Histidine, 84 g/L |
| | trehalose pH 5.5 |
| Rinse Volume (ml) | 31.6 |
| Rinse Concentration (g/L) | 53.2 |
| Rinse pH | 5.48 |
| Rinse Recovery (%) | 19.2 |

*The actual time was not recorded or could not be retrieved, it is based on a calculation of the flux and volume processed.

TABLE 4
Additional Development Excipient Concentration Results

| | Concentration | Histidine | Trehalose |
|---|---|---|---|
| Sample Name | (g/L) | (mM) | (g/L) |
| Diafiltration Pool | 109.4 | 27.81 | Not Tested |
| Concentration Load | 78.6 | 27.57 | 94.02 |
| Concentration 1 | 172 | 20.15 | 84.65 |
| Concentration 2 | 188 | 19.47 | 84.24 |
| Concentration 3 | 209 | 18.11 | 81.86 |
| Spike Buffer | N/A | 27.83 | 392.88 |

The method was scaled up to the 500 L pilot scale (Lot 12P126J603-MV-B): see Table 5 for process details. 517 g of Capto Adhere® purified material was concentrated to 107 g/l using a 0.5 m² Millipore® V-screen membrane, and then dia-filtered with 35 mM histidine, pH 5.29 buffer, a feed flow rate of 1000 LMH and a feed pressure of 40 psi. The retentate was then spiked with 4× trehalose solution (30 mM histidine, 400 g/l trehalose, pH 5.22), which was added directly into the to reservoir taking into account the system hold-up volume. The spiked material was then concentrated to 202 g/l, and the concentrated product removed from the system. The skid was rinsed with 20 mM histidine, 84 g/l trehalose, pH 5.50 buffer, and the rinse added to the concentrated material. The measured concentration of the final combined solution was 160 g/l with an overall yield of 97.1%.

Table 6 summarizes the excipient concentration and product quality results for the experiment, which shows that the final combined pool levels were within 10% of the aforementioned targeted concentrations, without any significant effect on product quality as measured by SEC when compared to past final UF values.

TABLE 5
Pilot Scale Process Data

| UFDF System and Load Charge | 12P120J603-MV-B |
|---|---|
| Equipment | Millipore System |
| Membrane Type | Millipore 30K V screen RC |
| Membrane Area, m2 | 0.5 |
| UF Protein Challenge (g/m²) | 1142 |
| Concentration 1 | |
| Load Volume (L) | 7.5 |
| Load Concentration (g/L) | 67.85 |
| Process Time (minutes) | 18 |
| Final Concentration (g/L) | 107 |
| Diafiltration | |
| Diafiltration Buffer | 35 mM histidine pH 5.3 |
| Diavolumes (TOV) | 8 |
| Feed Flow Rate (LMH) | ~1000 |
| Average Flux (LMH) | 20 |
| Diafiltration Time (hours) | 3.75 |
| Concentration 2 | |
| Spike Solution | 30 mM Histidine, 400 g/L |
| | trehalose pH 5.22 |
| Average TMP (psi) | <28 |
| Final flow Rate (LMH) | 108 |
| Process Volume (L) | 2.2 |
| Process Time (hr) | 1 |
| Product Concentration (g/L) | 202 |
| Product pH | 5.44 |
| Yield Recovery (%) | 85.7 |
| Rinse | |
| Rinse Buffer | 20 mM histidine, 84 g/L |
| | trehalose pH 5.5 |
| Rinse Volume (L) | 1.1 |
| Rinse concentration (g/L) | 53.7 |
| Rinse pH | 5.55 |
| Rinse Recovery (%) | 11.4 |

TABLE 6
Pilot Scale Excipient and Product Quality Results

| | Concentration | Histidine | Trehalose | Total | | Total |
|---|---|---|---|---|---|---|
| Sample Name | (mg/ml) | (mM) | (g/L) | HMMS | Monomer | LMMS |
| Diafiltered Pool | 107 | 28.28 | N/A | 1.0 | 99.0 | <0.1% |
| Post-Spike Pool | 78.6 | 28.04 | 90.35 | 0.8 | 99.2 | <0.1% |
| Concentration 2 Pool | 202 | 21.57 | 88.21 | 0.9 | 99.1 | <0.1% |
| Rinse Pool | 53.7 | 18.94 | 80.47 | 0.7 | 99.2 | <0.1% |
| Final Pool | 160 | 21.28 | 88.25 | 1.2 | 98.8 | <0.1% |
| DF Buffer | N/A | 34.80 | N/A | N/A | | |
| Excipient Buffer | | 31.52 | 413.75 | | | |
| Rinse Buffer | | 19.93 | 83.26 | | | |

Evaluation of Dia-Filtration Process

Protein Density and Viscosity at Different Concentrations

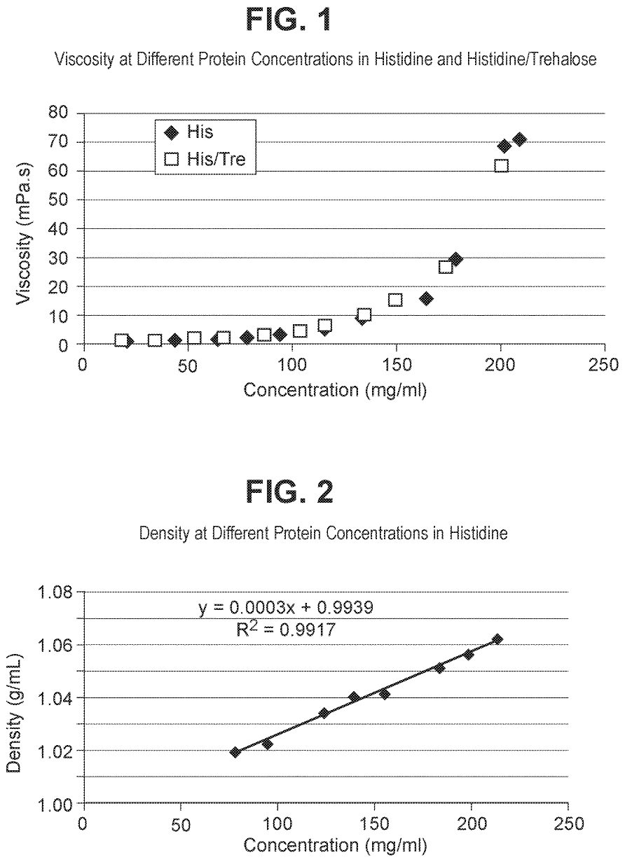

FIG. 1 plots the viscosity of bococizumab versus product concentration in (i) 20 to mM histidine, pH 5.5 and (ii) 20 mM histidine, 84 g/l trehalose, pH 5.5. The graph shows that at approximately 175 g/l the viscosity reaches the 30 cP value, which is considered the cutoff for viable UFDF processing at large scale.

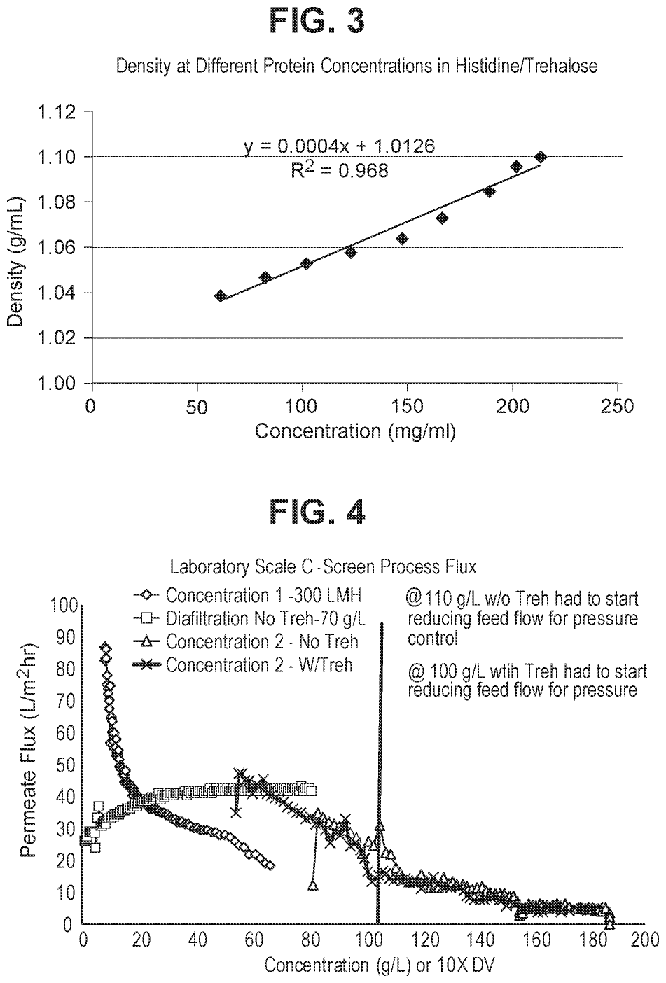

Densities of bococizumab in (i) 20 mM histidine, pH 5.5 and (ii) 20 mM histidine, 84 g/l trehalose, pH 5.5 solutions were measured and are shown in FIG. 2 and FIG. 3. The data shows that the density is slightly less in histidine buffer as compared to histidine/trehalose buffer, which is as expected.

Based on the experiments above, it was found that the targeted concentrations in the Drug Substance could be achieved at a manufacturing scale by with a DF buffer containing histidine, without trehalose.

The results from the experiments showed not only that the method of the invention resulted in acceptable yield, protein and excipient final concentrations, but also that it required lower protein concentration prior to the excipient spike, as compared to conventional methods (158 g/L versus 188 g/L). Such a lower protein concentration is easier to achieve on a regular basis as the process is scaled up. In addition, the method of the invention is advantageous over the conventional methods due to the better cost-of-goods profile achieved by removing trehalose from the diafiltration buffer.

It has been found that the UFDF process utilizing a Millipore® C-screen membrane, as an alternative to the Millipore® V-screen membrane, consistently resulted in a concentration greater than 175 g/l, which was sufficient to allow addition of the wash to pool (rinse) while still remaining above the 158 g/l needed prior to a 20x excipient spike. To ensure that the process would work with a trehalose spike, the process utilizing a C-Screen membrane was evaluated both at the laboratory and pilot scale.

The process was evaluated at the laboratory scale using Millipore® PLCTK C-Screen cassettes, utilizing the ultrafiltration (UF) run conditions outlined in Table 7.

For the TFF (Tangential flow Filtration) equipment, the lab scale process was performed employing a feed flow rate range of 30-300 LMH at an achievable pressure limit of approximately 50-55 psi as the operational limits. The upper feed flow rate of 300 LMH, where most of the process will occur, has a principle impact on process time, where reduced feed flow results in lower process flux which increases process pump time. The lower feed flow rate of 30 LMH is critical to the final concentration achievable, due to the increased viscosity increasing the pressure drop through the retentate channels, therefore lower flow rates enable pumping of more viscous solutions.

During the lab scale process run in the presence of approximately 84 g/l Trehalose a final concentration of 177 g/l was achieved in the final retentate pool with a feed flow rate of 30 LMH and feed pressure of 50 psi. The wash fractions from the lab scale runs were measured separately for yield as displayed in Table 8.

TABLE 7
Laboratory Scale Run Conditions

| | | Feed Pressure | | Retentate Pressure | | |
|---|---|---|---|---|---|---|
| Step | Solution | (psig) | | (psig) | | Target |
| Equilibration | 10 mM Histidine, 50 | 20 | (±2) | 10 | (±2) | ±0.2 pH Units |
| | mM NaCl, pH 6.4 | | | | | |
| Concentration 1 | VRF Product Pool | 34 | (±6) | 16 | (±6) | 600-1000 g/m² |
| Diafiltration | 35 mM Histidine pH | 34 | (±6) | 16 | (±6) | >7 TOV |
| | 5.3 | | | | | |
| Concentration 2 | Diafiltration Pool | 35 | (±20) | 10 | (±10) | 20-30% over DS |
| | | | | | | Target |
| Buffer Flush | 20 mM Histidine, 84 | 30 | (±20) | 10 | (±10) | Concentration |
| | g/L Trehalose, pH 5.5 | | | | | Dependent |

TABLE 8
Results of Laboratory Scale Development Experiments

| | | Conc. 1 Flux | Diafiltration Flux | Conc. 2 Flux | |
|---|---|---|---|---|---|
| | Load | (LMH) & | (LMH) & | (LMH) & | % Yield |
| | Challenge | Pressures | Pressures | Pressures | Ret/ |
| Experiment | (g/m²) | (P_{feed}/Pᵣₑₜ) | (P_{feed}/Pᵣₑₜ) | (P_{feed}/Pᵣₑₜ) | Flush |
| No | 354* | 90-20 | 25-42 | 35-4 | 85/13 |
| Trehalose | | (35/15) | (35/15) | (50/0) | |
| With | 354* | 20-90 | 25-42 | 45-4 | 85/13 |
| Trehalose | | (35/15) | (35/15) | (50/0) | |

*Lower load challenge is employed at lab scale due to material limitations and process cycling time and represents a worst case yield recovery option.

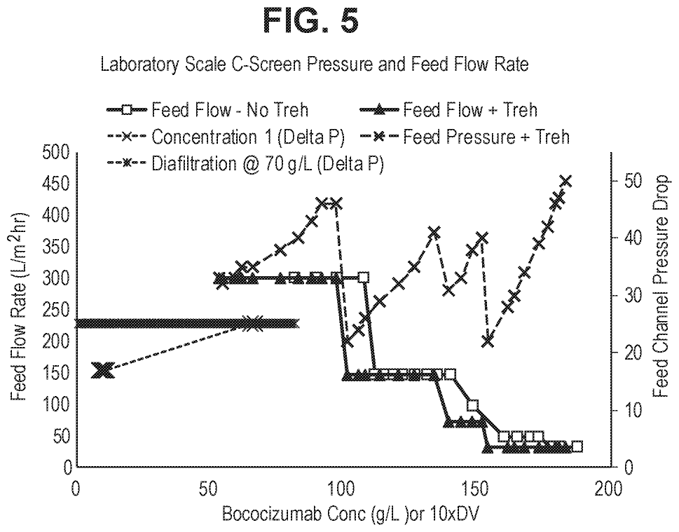

The laboratory scale process flux profile may be seen in FIG. 4, where the vertical line indicates the starting point for reducing the feed flow rate to keep the feed pressure below ˜50 psi with an open retentate (zero psi), where a reduction in process flux occurs due to reducing the cross flow rate. FIG. 5 shows the process feed channel pressure drop and the feed flow rate during the final concentration. In the lab scale system, the flow is manually adjusted by reducing the feed pump rate as the feed pressure approaches ˜50 psi.

Pilot Scale Confirmation Batches

The UFDF process with the C-screen membrane was performed at the 500 L scale to confirm that the final concentration targets could be achieved. The process is shown in Table 9 and the process data for the 3 lots performed in the pilot facility is shown in Table 10.

The results show that the process achieved high recoveries and that concentrations met the intermediate and final targets. In addition, the excipient concentrations for lot 13P120J604 were measured at 21.2 mM histidine, 85.4 g/l trehalose, and 0.051 g/l EDTA, which are within the target specifications of ±15%.

TABLE 9
UFDF Process Parameters for Pilot Scale Manufacturing

| Parameter | Details | Category |
|---|---|---|
| Membrane | Millipore PLCTK (30 kDa cellulose) C-Screen | |
| Membrane Surface Area | 500-1350 g/m² | Control Limit |
| Operating Temperature | 18-25° C. | Control Limit |
| Equilibration Buffer | 10 mM Histidine, 50 mM NaCl, pH 6.4 | |
| Pre-filter | ≤3000 L/m² for a 0.2 um filter | Target |
| Diafiltration Buffer | 35 mM Histidine pH 5.3 | |
| Dilution Buffer | 30 mM Histidine, 400 g/L Trehalose, pH 5.4 | |
| Final UF Flush Buffer | 20 mM Histidine, 84 g/L Trehalose, pH 5.5 | |
| Filter Conditioning/Equilibration | ≥10 L/m² | Target Range |
| Maximum Inlet & Retentate | ≤80 psig | Control Limit |
| Pressure | | |
| Concentration 1 Inlet Pressure | Setpoint 35 psig, target range 22-55 psig | Target Range |
| Concentration 1 Retentate | Setpoint 15 psig, target range 0-40 psig | Target Range |
| Pressure | | |
| Target Crossflow Rate | 0-10 L/min/m² | Target Range |
| Permeate Flux | 0-50 LMH | Expected |
| Diafiltration Inlet Pressure | Setpoint 40 psig, target range 22-55 psig | Target Range |
| Diafiltration Retentate Pressure | Setpoint 15 psig, target range 0-40 psig | Target Range |
| Diafiltration Concentration | 70-90 g/L based on tank volume of 350-450 L | Target |
| | 70-110 g/L | Target Range |
| Diafiltration Volume | Minimum 8 TOV's | Target Range |
| Permeate pH and Conductivity | After 8 TOVs verify pH is 5.5 ± 0.20 units. Continue | Control Limit |
| (Diafiltration End) | diafiltration until target is met. | |
| 4X Trehalose Buffer Addition | Add 30 mM Histidine, 400 g/L Trehalose pH 5.4 after DF | Control Limit |
| | at 1:3 ratio | |
| Concentration 2 Inlet Pressure | Setpoint 22-55 psig, target range 20-60 psig | Target Range |
| Concentration 2 Retentate | Setpoint 0 psig, target range 0-40 psig | Target Range |
| Pressure | | |
| Retentate Concentration | 170-190 g/L (per material balance in tank) | Target |
| | >158 g/L (actual retentate concentration) | Control Limit |
| Buffer Flush | minimal volume recirculated rinse | |
| UF Pool Concentration Target | 158 ± 10 g/L before 20X EDTA/PS80 Spike | Control Limit |
| UF Pool Density | Density calculation: 0.0004 * Concentration + 1.0126 | |
| Post-filter | ≤450 L/m² for a 0.2 um Filter | Target |

| | |
|---|---|
| Process Notes | Diafiltration Concentration based on 32-42 kg |
| | in 350-450 L retentate tank volume |
| | Add 4X trehalose buffer directly into tank after diafiltration. |
| | Addition at 1:3 ratio of total volume (volume in tank + system hold volume). |
| | Circulate for 10 minutes and concentrate to >158 mg/ml. |

TABLE 10
UFDF Process Data for 3 Pilot Scale Batches

| Batch # | UNITS | 13P120J604 | 13P120J605 | 13P120J606 |
|---|---|---|---|---|
| UF Filter Type | | 30 kD Millipore | 30 kD Millipore | 30 kD Millipore |
| | | C-screen | C-screen | C-screen |
| UF Total Area | m2 | 1.14 | 1.14 | 1.14 |
| Load Volume | L | 68.9 L/30.56 L | 81.53 L/26.57 L | 85.2 L/32.0 L |
| Load Concentration | g/L | 11.03 g/L/10.9 g/L | 11.04 g/L/11.22 g/L | 8.79 g/L/11.51 g/L |
| UF Protein Challenge | g/m2 | 959 | 950 | 982 |
| UF Volumetric Challenge | l/m2 | 87.2 | 95 | 103 |
| Concentration 1 Start Time | AM/PM | 08:21 | 08:21 | 09:08 |
| Concentration 1 End Time | AM/PM | 11:38 | 12:07 | 15:02 |
| Concentration 1 Permeate | L | 86.66 | 97.66 | 107.16 |
| Volume | | | | |
| Concentration 1 Feed Pressure | psig | 35 | 35 | 35 |
| Concentration 1 Retentate | psig | 15 | 15 | 15 |
| Pressure | | | | |
| Concentration 1 Permeate | psig | 0 | 0 | 0 |
| Pressure | | | | |
| Concentration 1 End Retentate | L | 12.8 | 10.4 | 10.2 |
| volume | | | | |
| Concentration 1 Average Flux | L/m2/h | 23.1 | 22.7 | 15.9 |
| Concentration 1 Average TMP | psig | 25 | 25 | 25 |
| Diafiltration Start Time | AM/PM | 11:49 | 12:14 | 10:05 |
| Diafiltration End Time | AM/PM | 14:45 | 16:22 | 13:39 |
| Diafiltration Permeate Volume | L | 109 | 82.76 | 103 |
| DiaVolumes | | 8.0 | 8.0 | 8.0 |
| Diafiltration Feed Pressure | psig | 40 | 40 | 40 |
| Diafiltration Retentate Pressure | psig | 15 | 15 | 15 |
| Diafiltration Permeate Pressure | psig | 0 | 0 | 0 |
| Diafiltration Average Flux | L/m2/h | 32.6 | 18.8 | 25.3 |
| Diafiltration Average TMP | psig | 27.5 | 27.5 | 27.5 |
| Concentration 2 Start Time | AM/PM | 15:15 | 16:45 | 14:02 |
| Concentration 2 End Time | AM/PM | 15:55 | 17:27 | 14:45 |
| Concentration 2 Feed Pressure | psig | 25-55 | 25-55 | 25-55 |
| Concentration 2 Retentate | psig | 0-2 | 0-2 | 0-2 |
| Pressure | | | | |
| Concentration 2 Permeate | psig | 0 | 0 | 0 |
| Pressure | | | | |
| Concentration 2 Permeate | L | 7.1 | 7.9 | 6.8 |
| Volume | | | | |
| Concentration 2 Average Flux | L/m2/h | 9.3 | 9.9 | 8.3 |
| Concentration 2 Average TMP | | 20 | 20 | 20 |
| (psig) | | | | |
| Product Pool Volume | L | 6.433 | 6.337 | 7.175 |
| Product Pool Concentration | g/L | 158.6 | 157.5 | 156.6 |
| Recovery | % | 95 | 92 | 99 |
| Wash Pool Volume | L | 833 | 915 | 790 |
| Wash Pool Concentration | g/L | 62.88 | 63.20 | 54.28 |
| Wash Pool Grams | g | 52 | 58 | 43 |
| Wash Pool Ratio | g/m2 | 5.14% | 5.79% | 3.82% |

A.3 Conclusions

The above-described experiments were able to demonstrate 92-99% step yield while achieving the Drug Substance target.

The Example demonstrates that a UFDF method according to the invention is suitable for the preparation of a highly concentrated (150 g/l) bococizumab Drug Substance with the pH and all excipient concentrations in the acceptable ranges. The same may be achieved with other proteins with the same benefits, especially with proteins having a particularly high viscosity.

### B—EXAMPLE 2

In illustrative Example 2, the protein of interest is antibody C1GM, an IL-7R antagonist monoclonal antibody that specifically binds to IL-7R. The method has been designed to achieve a targeted product concentration of 120 g/l in the Drug Substance, with the Drug Substance including the following excipients at a pH of 7.0:
histidine at a 20 mM concentration,
arginine at a 100 mM concentration,
sucrose at a 50 g/l concentration,
PS80 (PolySorbate 80) at a 0.02 g/l concentration, and
EDTA at a 0.5 g/l concentration.

It is deemed acceptable that the above requirements are achieved with a tolerance of ±10 g/l in the protein concentration, of ±15% in the excipients concentration and of ±0.5 in the pH value.

In terms of yield, the method is required to achieve a product recovery of more than 85%.

The starting material used for the experiments described below was a fully purified solution that had been processed through MabSelect® and Q membrane chromatography.

Ultrafiltration/Diafiltration Device

All experiments were performed using a GE Crossflow system (300 mL reservoir) or the Quattroflow198 pump system fitted with Pellicon 3® (30 KDa, C-screen, 88 cm²) regenerated cellulose membranes. Transmembrane pressure (TMP) was maintained at approximately 14-22 psi with P_{Feed}<55 psi. Unless otherwise specified, all rinses were generated by recirculating rinse buffer for >15 minutes, then concentrating to the minimal working volume of the system.

Analytical Assays

UV-visible spectrophotometry for protein concentration was performed using the Thermo Scientific Nanodrop 2000C™, or Solo VPE™ from C Technologies Inc. The extinction coefficient at 280 nm, as determined experimentally by ARD, is 1.51 mL*mg⁻¹*cm⁻¹.

Experiments

Experiment 1

The starting material was spiked with 5% of 2 M NaCl and adjusted to pH 7.0 with 2 M Tris base, concentrated to 50 g/L, diafiltered with 22 mM histidine, 110 mM arginine pH 7.0, spiked with 5× sucrose buffer (22 mM histidine, 110 mM arginine, 275 g/L sucrose pH 7.0), and concentrated to 146.9 g/L at a feed flow rate of ˜34 LMH, as detailed in Table 11. The pH of the concentrated solution was 7.00. The UF system was flushed in a single pass mode (without recirculation) with the diafiltration solution, resulting in a concentration of 33 g/L. The overall yield was approximately 88%.

TABLE 11
Diafiltration and Concentration with Arginine Buffer pH 7.0

| | |
|---|---|
| Notebook | 706263-77 |
| Pump | Quattro Flow |
| UF Protein Challenge (g/m²) | 348 |
| Concentration 1 | |
| Load Volume (L) | 1.36 |
| Load Concentration (g/L) | 2.25 |
| Feed Flow Rate (LMH) | Varied |
| Feed Pressure (psi) | <50 |
| Concentration 1 Time (minutes) | 320 |
| Concentration 1 Concentration | 49.8 |
| (g/L) | |
| Diafiltration | |
| Diafiltration Buffer | 22 mM histidine, 110 mM |
| | Arginine, pH 7.0 |
| DiaVolumes (TOV) | 8 |
| Feed Flow Rate @ DF (LMH) | 300 |
| Feed Pressure @ DF (psi) | 40 |
| TMP @ DF (psi) | 20 |
| Permeate Flow Rate @ DF (LMH) | 11.5-16.5 |
| Diafiltration Time (minutes) | 220 |
| Concentration 2 | |
| Spike Solution | 22 mM histidine, 110 mM |
| | Arginine, 275 g/L sucrose pH 7.0 |
| TMP @ Concentration 2 (psi) | <25 |
| Max Feed Pressure (psi) | 50 |
| Flow rate at End (LMH) | 34 |
| Concentration 2 Time (minutes) | Not recorded |
| Final Concentration (g/L) | 146.9 |

Tables 12-14 show the excipient, CGE and SEC assay results. The histidine, arginine, and sucrose concentrations in the final concentrated material were all within ±10% of the desired value. There was no new aggregation formed during the UF process, nor any change in the level of fragmentation.

TABLE 12
Process Excipient Concentrations for Arginine Buffer pH 7.0

| | Protein | | | |
|---|---|---|---|---|
| | Concentration | Arginine | Histidine | Sucrose |
| Sample Name | (g/L) | (mM) | (mM) | (g/L) |
| Diafiltrate | 50.1 | 113.7 | 22.6 | N/A |
| Post-Spike | 38.2 | 110.0 | 21.9 | 55.0 |
| Final | 146.9 | 108.5 | 19.8 | 50.0 |
| Concentrate | | | | |
| System Rinse | 33 | 111.1 | 22.1 | 49.9 |
| Diafiltration | N/A | 116.1 | 23.6 | N/A |
| Buffer | | | | |
| 5X Sucrose | N/A | 87.6 | 15.8 | 277.6 |
| Buffer | | | | |

TABLE 13
nrCGE and rCGE Results for Arginine Buffer pH 7.0 Experiment

| | nrCGE | | | rCGE | | |
|---|---|---|---|---|---|---|
| | | % | | | % | |
| | % | Frag- | % | % | Frag- | % |
| Sample | IgG | ment | Other | HC + LC | ment | Other |
| ANTI-IL-7R | 97.5 | 2.5 | 0 | 98.7 | 0.5 | 0.8 |
| Reference | | | | | | |
| Diafiltrate | 96.2 | 3.8 | 0 | 98.8 | 0.5 | 0.7 |
| Post-Spike | 96.2 | 3.8 | 0 | 99.1 | 0.3 | 0.5 |
| Final Concentrate | 96.3 | 3.7 | 0 | 98.9 | 0.5 | 0.6 |
| System Rinse | 96.8 | 3.2 | 0 | 99.1 | 0.3 | 0.6 |

TABLE 14
SEC Results for Arginine Buffer pH 7.0 Experiment

| Sample Name | % HMMS | % LMMS | % Monomer |
|---|---|---|---|
| Diafilitrate | 0.6 | 0.2 | 99.2 |
| Post-Spike | 0.6 | 0.2 | 99.2 |
| Final Concentrate | 0.6 | 0.2 | 99.2 |
| System Rinse | 0.6 | 0.2 | 99.2 |

Experiment 2

The experiment was repeated, as detailed in Table 15, the only difference being the way the spike volume was calculated. The total volume in the UF system prior to the spike was calculated from the overall material balance (the total load divided by the to diafiltrate concentration) versus adding the volume in the reservoir plus the system hold-up volume. After the 5X sucrose spike, the protein was concentrated to 183.6 g/L at ˜34 LMH feed flow rate and P_{feed}<50 psi.

TABLE 15
Diafiltration and Concentration with Arginine Buffer at pH 7.0

| | |
|---|---|
| Notebook | 706263-78 |
| Pump | Quattro Flow |
| UF Protein Challenge (g/m²) | 387 |
| Concentration 1 | |
| Load Volume (L) | 1.5 |
| Load Concentration (g/L) | 2.27 |
| Feed Flow Rate (LMH) | varied |
| Feed Pressure (psi) | <50 |
| Concentration 1 Time (minutes) | 400 |
| Concentration 1 Concentration | 43 |
| (g/L) | |
| Diafiltration | |
| Diafiltration Buffer | 22 mM histidine, 110 mM |
| | Arginine, pH 7.0 |
| DiaVolumes (TOV) | 10 |
| Feed Flow Rate @ DF (LMH) | 300 |
| Feed Pressure @ DF (psi) | 28 |
| TMP @ DF (psi) | 20 |
| Permeate Flow Rate @ DF (LMH) | 13.4-19.1 |
| Diafiltration Time (minutes) | 300 |
| Concentration 2 | |
| Spike Solution | 22 mM histidine, 110 mM Arginine, |
| | 275 g/L sucrose pH 7.0 |
| TMP @ Concentration 2 (psi) | <25 |
| Max Feed Pressure (psi) | 50 |
| Flow Rate at End (LMH) | 34 |
| Concentration 2 Time (minutes) | Not recorded |
| Final Concentration (g/L) | 183.6 |

Table 16 shows that the excipient concentrations, histidine, arginine, and sucrose concentrations in the final material were within ±10% of the desired target value. The difference in how the spike volume was calculated did not appear to have any significant effect on the final excipient concentrations.

TABLE 16
Excipient Concentrations for Arginine pH 7.0 Buffer

| | Protein | | | |
|---|---|---|---|---|
| | Concentration | Arginine | Histidine | Sucrose |
| Sample Name | (g/L) | (mM) | (mM) | (g/L) |
| Diafiltrate | 43.71 | 112.9 | 21.8 | ND |
| Post-Spike | 36.2 | 113.1 | 22.1 | 55.6 |
| Final Concentrate | 182 | 106.9 | 19.4 | 48.5 |
| 5X Spike Buffer | N/A | 102.1 | 22.2 | 266.7 |

Experiment 3

The experiment was repeated for a third time after the final formulation was nominated, and the results are detailed in Table . After diafiltration, the addition volume of the 5× spike solution was calculated as outlined in Experiment 2. The protein was concentrated to 190 g/L at ˜34 LMH feed flow rate and P_{feed}<50 psi. The UF system was rinsed with 20 mM histidine, 100 mM arginine, 50 g/L sucrose, pH 7.0 in the single pass mode. The protein concentration in the combined retentate and rinse pool was 151 g/L, resulting in an overall yield of approximately 84%.

TABLE 17
Diafiltration and Concentration with Arginine Buffer at pH 7.0

| | |
|---|---|
| Notebook | 706263-79 |
| Pump | Quattro Flow |
| UF Protein Challenge (g/m²) | 500 |
| Concentration 1 | |
| Load Volume (L) | 1.96 |
| Load Concentration (g/L) | 2.25 |
| Feed Pressure (psi) | <50 |
| Concentration 1 Time (minutes) | Not recorded |
| Concentration 1 Concentration | 45.4 |
| (g/L) | |
| Diafiltration | |
| Diafiltration Buffer | 22 mM histidine, 110 mM |
| | Arginine, pH 7.0 |
| DiaVolumes (TOV) | 8 |
| Feed Flow Rate @ DF (LMH) | 450 |
| Feed Pressure @ DF (psi) | 30 |
| TMP @ DF (psi) | 25 |
| Permeate Flow Rate @ DF (LMH) | 18 |
| Diafiltration Time (minutes) | 300 |
| Concentration 2A | |
| Spike Solution | 22 mM histidine, 110 mM |
| | Arginine, 275 g/L sucrose pH 7.0 |
| TMP @ Concentration 2 (psi) | <25 |
| Max Feed Pressure (psi) | 50 |
| Flow Rate at End (LMH) | 34 |
| Concentration 2 Time (minutes) | Not recorded |
| Final Concentration (g/L) | 190 |

Table 18 summarizes the excipient concentrations, showing that the histidine, arginine, and sucrose concentrations were all within ±10% of the desired value. Table 19 and Table 20 indicate that no additional aggregation or fragmentation was formed during the UFDF process.

TABLE 18
Excipient Concentrations for Arginine pH 7.0 Buffer

| | Protein | | | |
|---|---|---|---|---|
| | Concentration | Histidine | Arginine | Sucrose |
| Samples | (g/L) | (mM) | (mM) | (g/L) |
| Diafiltrate | 45.4 | 22 | 110.4 | N/A |
| Post-Spike | 37.3 | 22 | 110.9 | 55.5 |
| Concentration 2 | 190 | 19.5 | 102 | 49.7 |
| System Rinse | 37.6 | 20.5 | 102.2 | 52.2 |
| Final ANTI-IL-7R | 151.6 | 19.9 | 102.7 | 48.9 |
| Material | | | | |
| Diafiltration Buffer | N/A | 22.2 | 110.3 | N/A |
| 5X Spike Buffer | N/A | 22.2 | 110.4 | 274.4 |
| Rinse Buffer | N/A | 20 | 99.1 | 51.8 |

TABLE 19
nrCGE and rCGE Results for Arginine pH 7.0 Buffer

| | nrCGE | | | rCGE | | |
|---|---|---|---|---|---|---|
| | | % | | | % | |
| | % | Frag- | % | % | Frag- | % |
| Sample | IgG | ment | Other | HC + LC | ment | Other |
| ANTI-IL-7R | 97.1 | 2.9 | <0.3 | 98.7 | 0.5 | 0.8 |
| Reference | | | | | | |
| ANTI-IL-7R | 96.2 | 3.8 | <0.3 | 98.9 | 0.4 | 0.7 |
| diafiltrate | | | | | | |
| ANTI-IL-7R spike | 96.1 | 3.9 | <0.3 | 99 | 0.4 | 0.7 |
| ANTI-IL-7R | 95.2 | 4.5 | 0.3 | 99 | 0.3 | 0.8 |
| concentrate | | | | | | |
| ANTI-IL-7R rinse | 96.1 | 3.9 | <0.3 | 99 | 0.3 | 0.7 |
| ANTI-IL-7R final | 95.3 | 4.4 | 0.3 | 99 | 0.3 | 0.7 |

TABLE 20
SEC Results for Arginine pH 7.0 Buffer

| | % Total | % Total | % |
|---|---|---|---|
| Sample Name | HMMS | LMMS | Monomer |
| ANTI-IL-7R Std | 0.6 | 0.2 | 99.1 |
| ANTI-IL-7R | 0.5 | 0.3 | 99.3 |
| Diafiltrate | | | |
| ANTI-IL-7R Spiked | 0.5 | 0.3 | 99.3 |
| ANTI-IL-7R Con | 0.8 | 0.3 | 98.9 |
| ANTI-IL-7R Rinse | 0.5 | 0.3 | 99.3 |
| ANTI-IL-7R Final | 0.7 | 0.3 | 99 |

The process as performed in Experiment 3 above results in acceptable concentration values for all of the excipients and the protein of interest, and does not appear to have an effect on either formation of aggregate or fragmentation. This process will be scaled up to the pilot scale to ensure that it performs as expected.

Pilot Scale UFDF Process to The UF process developed above (Experiment 3) was tested in the Pilot Plant using a Millipore® C-screen regenerated cellulose membrane, and using material purified from a 500 L scale bioreactor.

During the unit operation, detailed in Table 21, 86 L of starting material at a starting product concentration of 2.92 g/L was spiked with 5% of 2 M NaCl, then concentrated to 44.6 g/L. The material was then diafiltered with 22 mM histidine, 110 mM arginine, pH 7.0 at a feed flow rate of approximately 150 LMH and P_{feed}<40 psi. After 8 TOV diafiltration, the retentate was spiked with 22 mM histidine, 110 mM arginine, 275 g/L sucrose pH 7.0, and recirculated for 10 minutes, then concentrated to 191.4 g/L. The concentration process was stopped at 30 LMH permeate flow rate and P_{feed}<50 psi. The skid was then rinsed with 20 mM histidine, 100 mm histidine, 50 g/L sucrose, pH 7.0 in single pass mode. The overall yield was approximately 87%. The entire UF process took approximately 5 hours to complete.

A UF pool at a concentration of 135.4 g/L was created by mixing the retentate pool, the rinse pool, and additional rinse buffer. The pool was filtered through a Millipore® 05/0.2 um Opticap Express SHC at 59 L/m² throughput. A 20× EDTA and PS80 excipient buffer was spiked into the UF pool to produce Drug Substance at a final concentration of 129.4 g/L.

TABLE 21
Pilot Scale UF Process Data

| UFDF System and Load Charge | |
|---|---|
| Membrane Type | Millipore 30K C screen RC |
| Membrane Area, m2 | 2.28 |
| UF Protein Challenge (g/m²) | 123 |
| Concentration 1 | |
| Load Volume (L) | 96 |
| Load Concentration (g/L) | 2.923 |
| Concentration 1 Time (minutes) | ~120 |
| Concentration 1 Concentration | 44.6 |
| (g/L) | |
| Diafiltration | |
| Diafiltration Buffer | 22 mM Histidine, 110 mM |
| | Arginine pH 7.0 |
| DiaVolumes (TOV) | 8 |
| Retentate Flow Rate @ DF (LMH) | 131.6 |
| Feed Pressure @ DF (psi) | 29 |
| TMP @ DF (psi) | 24 |
| Permeate Flow Rate @ DF (LMH) | 18.4 |
| Diafiltration Time (minutes) | 80 |
| Concentration 2 | |
| Spike Solution | 22 mM Histidine, 110 mM |
| | Arginine, 275 g/L |
| | Sucrose pH 7.0 |
| TMP @ Concentration 2 (psi) | 25 |
| Retentate Flow Rate @ End (LMH) | 30 |
| Concentration 2 Final Volume (L) | 0.738 |
| Concentration 2 Time (minutes) | ~30 |
| Concentration 2 Concentration | 191.4 |
| (g/L) | |
| Concentration 2 Recovery (%) | 50.35 |
| Rinse | |
| Rinse Buffer | 20 mM Histidine, 100 mM |
| | Arginine, 50 g/L Sucrose |
| | pH 7.0 |
| Rinse Final Volume (mL) | 1057 |
| Rinse Concentration (g/L) | 97.3 |
| Rinse Recovery (%) | 36.7 |

The excipient concentrations are summarized in Table 22, which shows that the histidine, arginine, and sucrose concentrations in the final pool were within ±15% of the target values. The targeted range during the process development at lab scale was set at ±10% of the target values for all excipient concentrations, but the acceptance range at large scale was set at ±15% to allow for latitude during scale-up. Table 23 and Table 24 summarize the product quality results from the run, which show that no increase in aggregation or fragmentation was detected.

TABLE 22
Pilot Scale Run Excipient Concentration Results

| | Concentration | Arginine | Histidine | Sucrose |
|---|---|---|---|---|
| Sample Name | (g/L) | (mM) | (mM) | (g/L) |
| Diafiltration Pool | 44.6 | 111.4 | 22.2 | N/A |
| 5X Spiked Pool | 32.9 | 111.2 | 22.1 | 46.3 |
| Concentration 2 | 191.4 | 105.8 | 20.1 | 43.9 |
| Retentate | | | | |
| Final Rinse Pool | 97.3 | 101.8 | 19.9 | 46.4 |
| Drug Substance | 135.4 | 103.9 | 20.2 | 43.8 |
| UF Buffer | N/A | 110.6 | 22.2 | N/A |
| 5X Spike | N/A | 112.7 | 22.5 | 269.5 |
| Rinse Buffer | N/A | 101.0 | 20.4 | 46.9 |

TABLE 23
Pilot Scale Run SEC Results

| | % Total | % Total | % |
|---|---|---|---|
| Sample Name | HMMS | LMMS | Monomer |
| ANTI-IL-7R Reference | 0.6 | 0.4 | 99.0 |
| UF Load | 0.5 | 0.4 | 99.2 |
| Concentration 2 UF | 0.7 | 0.4 | 98.9 |
| Retentate | | | |
| Final Rinse Pool | 0.6 | 0.3 | 99.1 |
| Drug Substance | 0.6 | 0.3 | 99.0 |

TABLE 24
Pilot Scale Run nrCGE Results

| Sample | % IgG | % Fragment | % Other |
|---|---|---|---|
| ANTI-IL-7R Reference | 96.5 | 3.1 | 0.3 |
| UF Load | 96.7 | 3.3 | 0.0 |
| Diafiltration Pool | 97.3 | 2.7 | 0.0 |
| Concentration 2 UF | 96.2 | 3.4 | 0.3 |
| Retentate | | | |
| Final Rinse Pool | 97.1 | 2.9 | 0.0 |
| Drug Substance | 96.5 | 3.5 | 0.0 |

Conclusions

In conclusion, the above-described experiments demonstrate the successful process to development of a UFDF process for >120 mg/ml drug substance for the ANTI-IL-7R antibody of interest. The UFDF process includes an initial concentration, a diafiltration, a sucrose spike prior to a final concentration, then spiking with the remaining excipients. The pH and all excipient concentrations in the developed process are in the acceptable ranges.

1. A method of preparing a protein formulation including excipients and at least one therapeutic protein, the method comprising the sequential steps of:
(a) providing a solution comprising said protein;
(b) concentrating the protein in the solution by a first ultra-filtration step;
(c) diafiltering the solution thus obtained with a diafiltration buffer including at least one first excipient, whereby a retentate is obtained comprising the protein and the first excipient;
(d) adding a second excipient to the retentate obtained from the diafiltration step;
(e) further concentrating the protein in the retentate by a second ultra-filtration step in an ultra-filtration equipment; and
(f) adding at least one final excipient, whereby the protein formulation with a desired protein concentration and including said first and final excipients is obtained.

2. The method of claim 1, further including, after step (e) and before step (f), rinsing the ultra-filtration equipment with a rinse buffer, whereby the recovery of the protein is enhanced.

3. The method of claim 2, wherein the rinse buffer comprises the first and the second excipients at concentrations substantially equal to, respectively, the concentrations of the first and of the second excipients in the protein formulation.

4. The method of claim 1, wherein the first excipient is an amino-acid, preferably histidine.

5. The method of claim 1, wherein the first excipient in the protein formulation has a concentration of between 16 and 24 mM, preferably of between 17 and 23 mM, most preferably of about 20 mM.

6. The method of claim 1, wherein the second excipient is a sugar, preferably a disaccharide.

7. The method of claim 1, wherein the final excipients include a surfactant, preferably polysorbate 80.

8. The method of claim 1, wherein the final excipients include a chelating agent, preferably EDTA.

9. The method of claim 1, wherein the protein formulation has a protein concentration of between 110 and 165 g/l.

10. The method of claim 1, wherein the protein is an antibody.

11-33. (canceled)

34. The method of claim 10, wherein the antibody is an anti-IL-7R antibody.

35. The method of claim 10, wherein the antibody has a VH region comprising the amino acid sequence shown in SEQ ID NO 13, and VL region comprising the amino acid sequence shown in SEQ ID NO 14.

36. The method of claim 34, wherein the protein formulation has a protein concentration of between 110 and 130 g/l, preferably of about 120 g/l.

37. The method of claim 34, wherein the second excipient in the protein formulation is sucrose at a concentration of between 42 and 58 g/l, preferably of about 50 g/l.

38. The method of claim 34, wherein the final excipients include polysorbate 80 which, in the protein formulation, has a concentration of between 0.017 and 0.023 g/l, preferably of about 0.02 g/l.

39. The method of claim 34, wherein the final excipients include EDTA which, in the protein formulation, has a concentration of between 0.42 and 0.58 g/l, preferably of about 0.5 g/l.

40. The method of claim 34, wherein the final excipients include arginine which, in the protein formulation, has a concentration of between 85 and 115 mM, preferably of about 100 mM.

41. The method of claim 34, wherein the protein formulation has a pH of between 6.5 and 7.5, preferably of about 7.0.

42. The method of claim 34, wherein the solution provided in step (a) has a protein concentration of between 2.6 and 3.4 g/l, preferably of about 3 g/l.

43. The method of claim 34, wherein the protein is concentrated to between 36 and 54 g/l, preferably to between 40 and 50 g/l, and most preferably to about 45 g/l, by the first ultra-filtration step.

44. The method of claim 34, wherein the protein is concentrated to between 170 and 210 g/l, preferably to about 190 g/l, by the second ultra-filtration step.

45. The method of claim 34, wherein the first excipient in the diafiltration buffer has a concentration higher than the concentration of the first excipient in the protein formulation, said concentration of the first excipient in the diafiltration buffer being preferably of between 19 and 25 mM, most preferably of about 22 mM.

46. The method of claim 34, wherein the diafiltration buffer includes arginine at a concentration of between 95 and 125 mM, preferably of about 110 mM.

47. The method of claim 34, wherein the diafiltration buffer has a pH of between 6.5 and 7.5, preferably about 7.0.

48. The method of claim 34, wherein adding the second excipient to the retentate obtained from the diafiltration step is achieved by adding a first additive solution to the retentate, said first additive solution comprising the second excipient at a concentration of between 230 and 320 g/l, preferably of about 275 g/l.

49. The method of claim 48, wherein the first additive solution comprises the first excipient at a concentration substantially equal to the concentration of the first excipient in the diafiltration buffer and higher than the concentration of the first excipient in the protein formulation, said concentration of the first excipient in the first additive solution being preferably of between 19 and 25 mM, most preferably of about 22 mM.

50. The method of claim 48, wherein the first additive solution further comprises a final excipient.

51. The method of claim 50, wherein the first additive solution comprises about 22 mM histidine, 110 mM arginine and about 275 g/l sucrose, at a pH of about 7.0.

52. The method of claim 34, wherein adding the first additive solution to the retentate is performed at a dilution ratio of about 5, whereby one volume of the first additive solution is added to approximately 4 fold the same volume of the retentate.

53. The method of claim 34, wherein adding the final excipients includes the step of adding a second additive solution to the solution obtained from the second ultra-filtration step, said second additive solution comprising EDTA and polysorbate 80.

54. The method of claim 53, wherein adding the second additive solution is performed at a dilution ratio of about 20, whereby one volume of the second additive solution is added to approximately 19 fold the same volume of to the solution obtained from the second ultra-filtration step.

55. A formulation of an antibody having a high viscosity produced by the method of claim 1.

56. (canceled)

57. The formulation of claim 55, wherein the antibody is an anti-IL-7R antibody and wherein the protein formulation comprises:
from 110 g/l to 130 g/l, preferably about 120 g/l, of the anti-IL-7R antibody;
from 17 mM to 23 mM, preferably about 20 mM, of histidine;
from 42 g/l to 58 g/l, preferably about 50 g/l, of sucrose; and
from 0.017 g/l to 0.023 g/l, preferably about 0.02 g/l, of polysorbate and has a pH of between 6.5 and 7.5, preferably about 7.0.
